(19) United States
(12) Patent Application Publication
(10) Pub. No.: US 2002/0143268 A1
(43) Pub. Date: Oct. 3, 2002

(54) Bone implant testing

(76) Inventors: Neil Meredith, Harrogate (GB); Per Anders Petersson, Goteborg (SE)

Correspondence Address: NIXON & VANDERHYE P.C., 8th Floor, 1100 North Glebe Road, Arlington, VA, 22201-4714, US

(21) Appl. No.: 09/824,563

(22) Filed: Apr. 3, 2001

(51) Int. Cl. A61B 5/00

(52) U.S. Cl. 600/552

(57) ABSTRACT

A transducer assembly (10, 20, 80, 90) is used with an apparatus (100, 200) for detecting the stability of a bone implant (30). The transducer assembly uses one or more transducers to excite vibrations in said bone implant and to detect a response to the vibrations. A coupling (80) is used to detachably connect the one or more transducers to circuitry for driving the vibrations and detecting the response. A memory device (90) is provided within the transducer assembly. The memory device (90) can store one or more parameters read from outside of the transducer assembly via the coupling for controlling the use of the transducer.

### BACKGROUND OF THE INVENTION

1. Field of the Invention

This invention relates to the field of bone implant testing.

Bone implants are formed by the insertion of a fixture into a bone, typically the jaw bone of a human or animal subject. These bone-anchored fixtures are typically made of a metal such as titanium and they are frequently used in dentistry for attaching false teeth. bridges and prostheses. Such implants can also be used in cranio-facial econstruction.

The stability of an implant depends on the stiffness and strength of its bond with the surrounding bone. The bond will initially be weak after insertion of the implant but should gradually strengthen as the bone heals and integrates with the implant at their interface. Failure of the implant can be caused by improper insertion of the implant or by premature loading. Even once the bone has healed, subsequent overloading can cause the bone to migrate away from the implant, thus causing it to fail. Failure of these implants can be difficult to predict.

2. Description of the Prior Art

Known quantitative diagnostic techniques for predicting the performance of an implant at the time of placement and at subsequent times during the lifetime of the implant are described in GB-A-2 270 980 and WO-A-92/18053. The techniques described therein involve determining a resonant frequency characteristic of the stiffness of the implant/bone interface and monitoring any shifts in the resonant frequency as a function of the time since implantation.

A disadvantage of these known techniques is that a measured resonant frequency reflects not only the resonant frequency of the implant/bone interface, but is also affected by the type of bone implant on which the measurement is made and how the vibration measurement device is attached to the implant. Variations in the sensitivity of the receivers between different vibration measurement devices also affects the measurement. Therefore it is necessary to adjust the measured resonant frequency to take account of these unwanted variable factors. The adjustment of the measured resonant frequency is defined by a set of calibration parameters specific to a particular measurement device. A reliable quantitative diagnosis of implant stability depends on the accuracy of these calibration parameters. Errors in the calibration parameters can arise due to the clinician performing an unreliable calibration or as a result of using the wrong calibration parameter set. Furthermore damage/wear to a given vibration measurement device as a result of clinical use means that a set of calibration parameters, even if provided by the manufacturer, are only valid for a limited number of uses of the device.

### SUMMARY OF THE INVENTION

Viewed from one aspect the present invention provides a transducer assembly for use with an apparatus for detecting the stability of a bone implant, said transducer assembly comprising:

one or more transducers operable to excite vibrations in said bone implant and to detect a response to said vibrations;

a coupling for detachably connecting said one or more transducers to circuitry for driving said vibrations and detecting said response; and

a memory device within said transducer assembly operable to store one or more parameters to be read outside of said transducer assembly via said coupling for controlling use of said transducer assembly.

The invention recognises that the likelihood of inaccuracies in bone implant stability measurements can be reduced by providing a memory device as part of the transducer assembly and storing parameters that control the use of the transducer assembly on the memory device. The control parameters can be programmed onto the transducer assembly during the manufacturing process.

The calibration parameter(s) set for a transducer assembly could be stored separately in a computer file or on paper, but it is preferred that the calibration parameters for the bone/implant stability measurement are stored on the memory device. This reduces the likelihood of the wrong calibration parameter(s) being used and eliminates the need for the user to perform the calibration measurements.

Although a transducer assembly could be identified by a mark or a serial number stamped on a visible external surface, it is preferred that information to identifv the transducer assembly is stored on the memory device of the transducer assembly. This has the advantage that the identification information can be automatically logged against each implant stability measurement so that the clinician can elect to use the same transducer assembly for measurement on a given patient at different times and comparisons can be made on sets of results obtained from different transducer assemblies.

Whilst the transducer assembly could be used indefinitely without monitoring the number of uses, it is advantageous to store a connection count on the memory device of the transducer assembly to specify the number of times that has been connected to the signal source and the signal receiver. The value of the connection count can be monitored to give an indication of the reliability of the results obtained from the transducer assembly. As the connection count increases, the reliability of the measurements will typically deteriorate due to wear and tear on the components of the transducer assembly.

Although the connection count stored on the memory could be incremented each time transducer assembly is connected to the signal source and the signal receiver, it is preferred that a time value is stored corresponding to each increment of the connection count. This allows the user to monitor the frequency of use of a given transducer assembly.

Furthermore the time information associated with the connection count can be used to conditionally increment the counter. The counter can be incremented on the condition that the current time exceeds the time since the last connection by more than a predetermined threshold. This allows a given transducer assembly to be removed and repositioned on a patient during a single measurement session without incrementing the counter unnecessarily. Thus, the counter can be incremented once for each autoclaving of the transducer assembly.

It will be appreciated that the user of the transducer assembly could monitor the connection count and exercise judgement as to when the transducer assembly has been used too often. However, preferred operation is achieved when the transducer assembly is disabled when the connection count exceeds a predetermined threshold. This prevents the user from attempting to extend the lifetime of the transducer assembly for the purposes of economy at the expense of producing inaccurate implant stability measurements.

Viewed from another aspect the invention provides an apparatus for detecting the stability of a bone implant, said apparatus comprising:

a transducer assembly for use with an apparatus for detecting the stability of a bone implant, said transducer comprising:

one or more transducers operable to excite vibrations in said bone implant and to detect a response to said vibrations;

a coupling for detachably connecting said one or more transducers to circuitry for driving said vibrations and detecting said response; and

a memory device within said transducer assembly operable to store one or more parameters to be read outside of said transducer assembly via said coupling for controlling use of said transducer assembly; and

an instrument for use with said transducer assembly, said instrument comprising:

a memory reading circuit operable to read one or more parameters from a memory device within a transducer assembly; and

a variable frequency signal source operable to drive excitation vibrations in said bone implant via said transducer assembly;

a frequency response analyser operable to analyse a received response signal from said transducer assembly and to generate a frequency response of said bone implant to said excitation vibrations; wherein

at least one of said variable frequency signal source and said frequency response analyser operate in dependence upon said one or more parameters.

Viewed from a further aspect the invention provides a method for detecting the stability of a bone implant, said method comprising the steps of:

exciting vibrations in said bone implant using one or more transducers of a transducer assembly and detecting a response to said vibrations;

driving said one or more transducers and detecting said response by detachably connecting said transducer assembly to circuitry via a coupling of said transducer assembly; and

storing one or more parameters in a memory device within said transducer assembly wherein said parameters are to be read outside of said transducer via said coupling and employing said parameters to control the use of said transducer assembly.

### BRIEF DESCRIPTION OF THE DRAWINGS

The above, and other objects, features and advantages of this invention will be apparent from the following detailed description of illustrative embodiments which is to be read in connection with the accompanying drawings, in which:

FIG. 1 is a schematic diagram of an apparatus for performing dental implant stability measurements according to an example embodiment of the invention;

FIG. 2 is a schematic diagram of the transducer device of FIG. 1 attached to a bone implant;

FIG. 3A is a graph of an input frequency signal fed to the exciter transducer of the transducer device;

FIG. 3B is a graph of an output frequency signal generated by the receiver transducer of the transducer device;

FIG. 3C is a graph of the amplitude in dB of the vibrations detected by the receiver transducer against the excitation frequency in kHz;

FIG. 4A is a schematic diagram showing the transducer assembly of FIG. 1 in more detail;

FIG. 4B is a schematic diagram showing the EPROM device of the transducer plug of FIG. 4A and its interface with the instrument of FIG. 1; and

FIG. 5 is a flow diagram that demonstrates an example implant stability measurement process.

### DESCRIPTION OF THE PREFERRED EMBODIMENTS

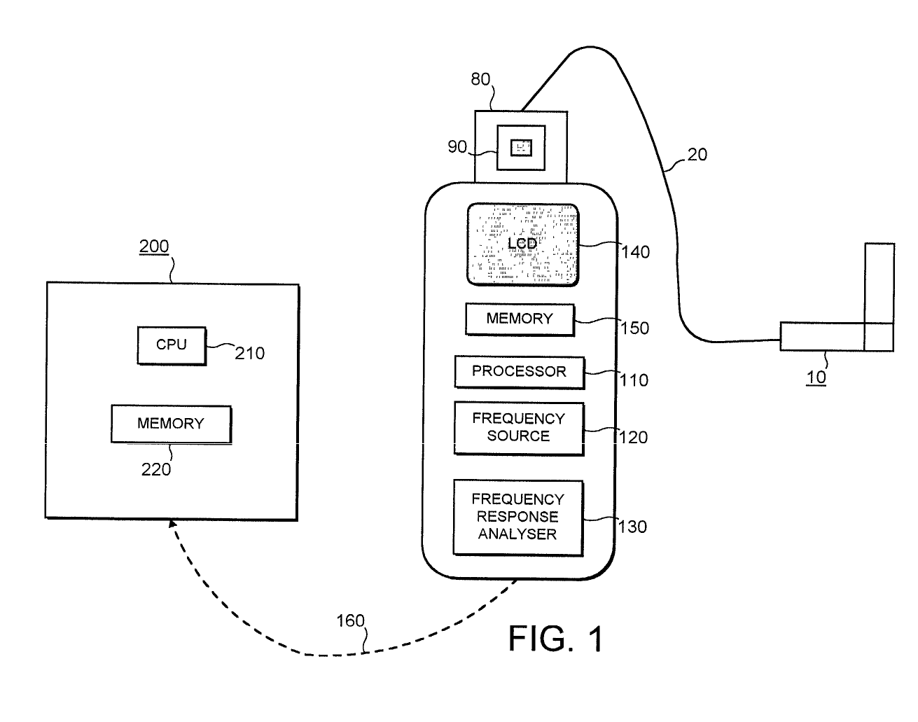

FIG. 1 is a schematic diagram of an apparatus for performing dental implant stability measurements. The stability measurement apparatus comprises a transducer device 10 which is formed by an L-shaped cantilever beam on which an exciter transducer and a receiver transducer are mounted. The transducer device 10 is attached to an instrument 100 via a cable 20 and a transducer plug 80. The transducer plug 80 houses an erasable programmable read only memory (EPROM) device 90 that is used to store information that controls the use of the transducer device. The EPROM 90 is typically first programmed (initialised) during the manufacture of the transducer device.

The instrument 100 comprises a microprocessor 110, a frequency source 120, a memory 150, a frequency response analyser 130 and a liquid crystal display screen 140. The frequency source 120 is a variable frequency AC signal generator capable of delivering an excitation voltage to the exciter transducer of the transducer device 10 via the cable 20. The frequency response analyser 130 is operable to detect and amplify an output signal from the receiver transducer of the transducer device corresponding to the vibrations induced in the cantilever beam by the exciter transducer. The output from the frequency response analyser typically represents the ratio of the response voltage to the excitation voltage. A resonance frequency of the structure in which vibrations are being excited will result in a peak in this ratio. The output from the frequency response analyser 130 is fed to the microprocessor 110 which is used to control the frequency output of the frequency source 120 and to store results of the measurements in the memory 150. The results of each measurement are displayed on the LCD screen 140.

The instrument 100 is coupled to a computer 200 via an infra-red link 160. Alternatively, the instrument 100 can be coupled to the computer 200 via a wire link, for example using an RS232 interface. Results can be downloaded from the instrument 100 to the computer 200. The computer 200 comprises a central processing unit (CPU) 210 and memory 220. The memory 220 is typically used to store implant stability measurement data for a group of patients over an extended timescale, for example, implant stability measurements may be made on each patient at 3 month intervals over a period of 12 months to monitor the stability of the implant. The results downloaded from the instrument 100 to the computer 200 can be compared against a database of implant stability measurements and contrasted with measurements for specific implant types at particular times after implantation. Software on the PC 200 is provided for performing the data analysis.

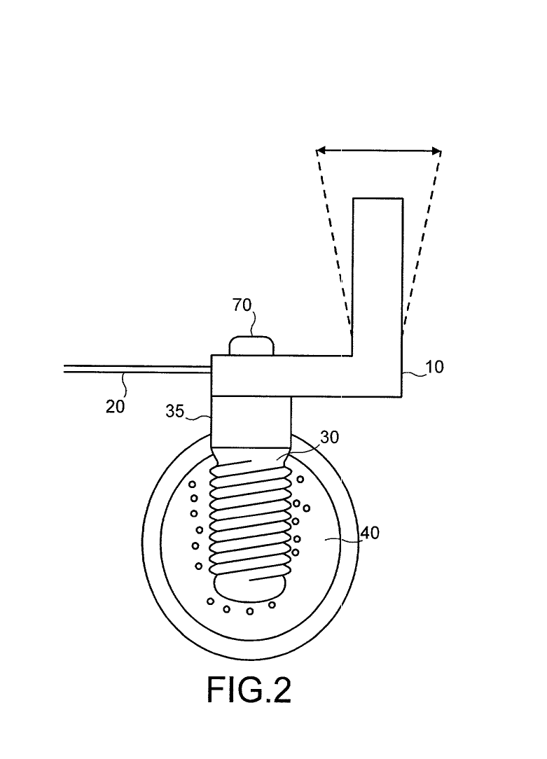

FIG. 2 is a schematic diagram of the transducer device attached to an implant. The bone implant comprises an implant fixture 30 which is screwed into the bone 40 and an implant abutment 35 of predetermined length which is attached to the implant fixture 30. The top of the fixture 30 is generally above the top of the bone 40. The implant abutment 35 takes the assembly above gum level. The implant abutment 35 is used for attachment of a prosthesis such as a tooth and is typically fitted at a time subsequent to the insertion of the implant fixture 30.

The transducer device 10 can be attached either to the implant abutment 35 as shown in FIG. 2 or directly to the implant fixture 30. The implant stability measurement will typically be performed with the transducer device 10 attached directly to the implant fixture 30 immediately after implantation and at initial followup tests, whereas it will be attached to the implant abutment 35 at subsequent stages when the abutment and prosthesis have been fitted. Due to differences in geometry, the transducer device that is attached to the implant abutment 35 is necessarily different from the transducer device that is used for direct attachment to the implant fixture 30.

When the abutment is first placed the implant stability test can be performed successively with a fixture level transducer and an abutment level transducer. It is important that implant stability measurements for a given patient made prior to and following the attachment of the implant abutment 35 can be reliably compared. To achieve this the measured ISQ value should be independent of whether the transducer is attached to the implant fixture 30 or to the implant abutment 35. The transducer device 10 is preferably attached to the implant abutment 35 or to the implant fixture 30 using a screw 70. However a push-fit connector or a clip attachment could be used as alternatives to the screw 70.

The implant fixture 30 is lodged in position in a section of bone 40 of a patient, typically a human jaw bone and the implant abutment 35 may be attached to the implant fixture 35. The transducer device 10 is attached to either the implant fixture 30 or the implant abutment 35. Vibrations are excited in the cantilever beam of the transducer device 10 by a swept frequency excitation signal delivered via the cable 20.

The vibration of the upright portion of the L-shaped cantilever beam is indicated by the arrow on FIG. 2. The excitation signal delivered to the cantilever beam will typically be in the frequency range from 3000 Hz to 20000 Hz. Secondary displacement in the implant resulting from the excitation is typically very small, being the order of microns. This means that the patient typically experiences no discomfort as a result of the implant excitation.

The response of the combined structure comprising the cantilever beam and dental implant to the excitation signal can be characterised by at least one resonance frequency. The value of the resonance frequency depends on the rigidity of the interface between the implant 30 and the bone 40. The rigidity of the interface will be a function of time since it will change as “osseointegration” occurs whereby bone heals around the embedded portion of the implant. The transducer device is such that the resonance frequency of the cantilever beam reflects the stiffness relative to the bone of the combined system comprising the bone implant 30 and the transducer device 10. The stiffness of the connection between the transducer assembly and the implant is arranged to be substantially constant by appropriately adjusting the torque on the screw attachment 70 so that it is much stiffer than the interface between the bone and the implant. The torque applied to the screw attachment 70 will typically be controlled to lie within a predetermined range of the order of 10 to 15 Ncm. Thus the resonance frequency measured by a receiver on the transducer device 10 reflects the stiffness of the interface between the implant and the bone.

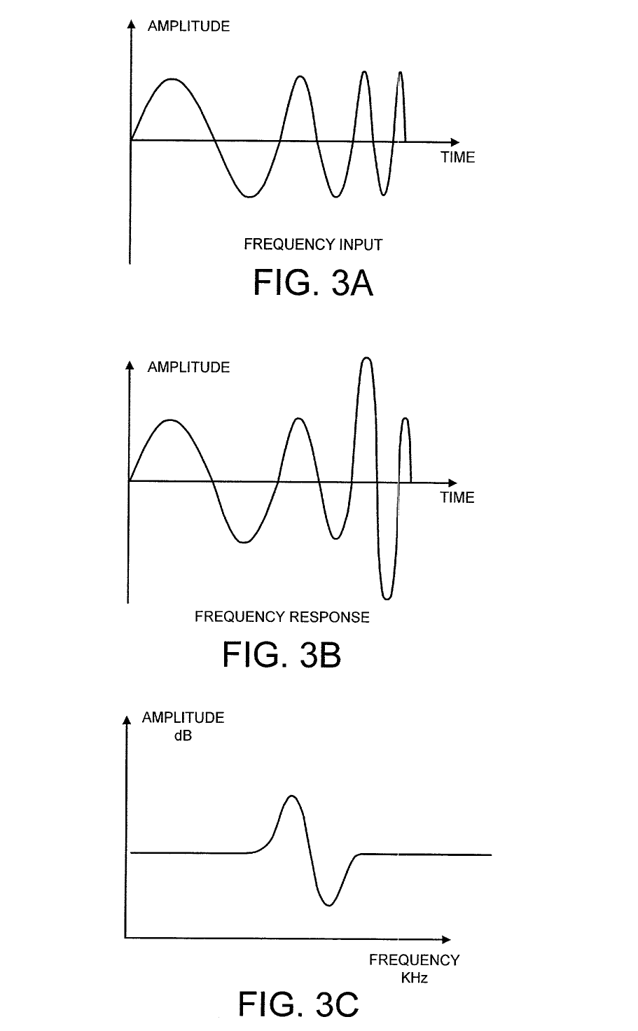

FIG. 3A shows a typical sinusoidal voltage excitation signal as applied to the cantilever beam of the transducer device 10. The frequency of the excitation signal is increased with time under the control of the microprocessor 110. FIG. 3B shows an example frequency response of the combined cantilever beam and implant structure of FIG. 2 to the excitation signal of FIG. 3A. The frequency response is characterised by a pronounced increase in amplitude in a particular time interval. This increase in amplitude corresponds to the resonance frequency characteristic of the implant/bone interface. FIG. 3C shows an example output of the frequency response analyser 120. It is a graph of amplitude in dB against frequency in kHz. The amplitude in dB is obtained from the ratio of the received voltage Vₒᵤₜ to the excitation voltage Vᵢₙ as defined by the formula 20 log₁₀ Vₒᵤₜ/Vᵢₙ. The resonant frequency corresponds to the frequency at which the peak amplitude occurs.

An important factor in the reliability of the implant testing method according to embodiments of the invention is that the resonance frequency obtained from the frequency response analyser 130 should be substantially equal for measurements on different implants with the same stiffness at the bone-implant interface. The measured resonance frequency should also be independent of whether or not an abutment is used to attach the transducer device to the implant and be suitably adjusted for any geometrical changes in the transducer device such as those changes that may occur due to wear and tear or due to variability between transducer devices resulting from the manufacturing process. To take account of the variations in the frequency response measured by the transducer device 10 arising from factors other than the stiffness of the implant/bone interface, an implant stability quotient (ISQ) is used. The ISQ is calculated by compensating the measured resonance frequency for differences in implant type, abutment lengths and variations in frequency response of the transducer devices. The ISQ is defined by the following equation:

ISQ=F_{R}×(k+n×L)+m×L+p,

where F_{R} is the measured resonance frequency; k and n are calibration factors in the range −1 to +1 with a resolution of 2/32768; m and p are calibration factors in the range −100 to +100 with a resolution of 1; and L is a predetermined abutment length which is zero if an abutment is not used.

The calibration factors are obtained during manufacture by testing the frequency response of each transducer device 10 when attached to aluminium calibration blocks. A group of calibration blocks is used, each calibration block having a different stiffness hence a different ISQ. The ISQ values for the calibration blocks are known prior to calibration of the transducer devices In the case where no abutment is used, L is 0; thus: ISQ=F_{R}×k+p and the factors k and p are determined by finding a “best fit” to a straight line of the calibration block ISQ value against the corresponding measured resonance frequency.

For the implant abutment transducer device the calibration is again performed by attaching it in turn to the set of calibration blocks having different predetermined ISQ values. In this case different lengths of abutment are tested with the transducer device for each calibration block. For a fixed abutment length, let A=k+n×L, and B=m×L+p. then:ISQ =F_{R}×A+B. The factors A and B are determined for each abutment length L, by establishing a best fit to a straight line of the known ISQ value of the calibration block against the corresponding measured resonance frequency. The factors k and n are determined by finding a best fit straight line to a plot of A against the abutment length L. Similarly, the factors m and p are determined by finding a best fit straight line to a plot of B against the abutment length L.

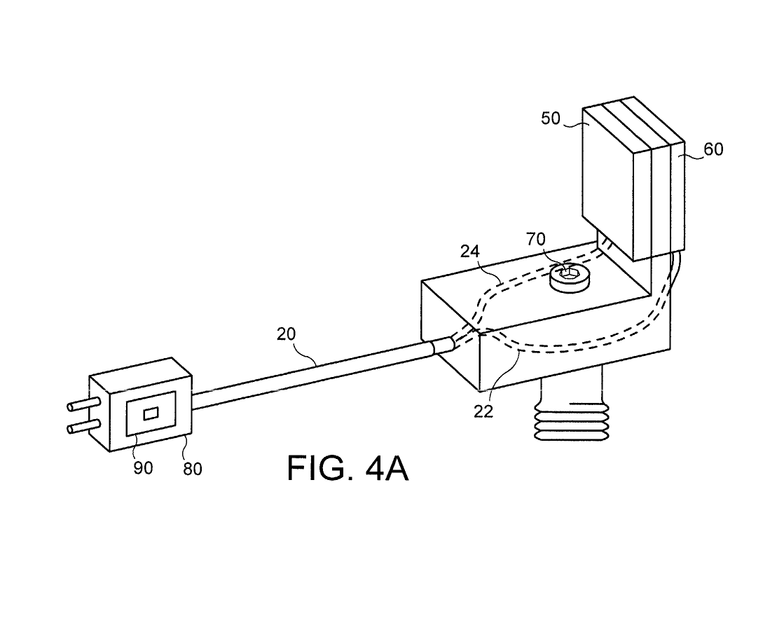

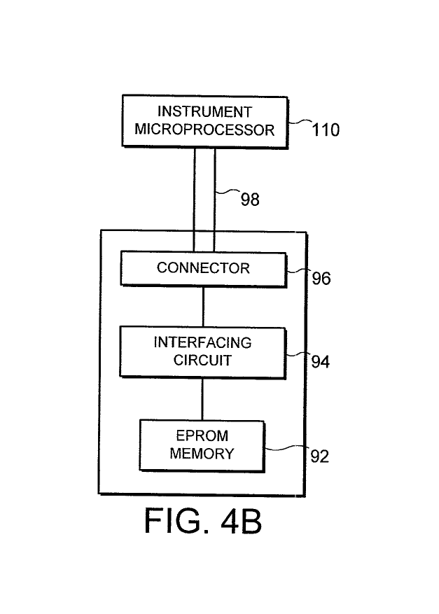

FIG. 4A is a schematic diagram showing the detailed structure of the transducer arrangement comprising the transducer device 10, the cable 20, the transducer plug 80 and the EPROM device 90. This figure shows an exciter transducer 50 and a receiver transducer 60 which are bonded to opposite sides of the upright portion of the cantilever beam. The cantilever beam is L-shaped in this embodiment of the invention but alternative embodiments could have a cantilever beam of a different shape, for example, it could be straight or U-shaped. The exciter transducer 50 and the receiver transducer of this embodiment are piezoelectric elements. It will be appreciated that the exciter and/or receiver transducer could alternatively comprise strain gauges, electromagnetic devices or devices employing sonic resonance.

In an alternative embodiment of the invention a single transducer element is used both as a vibration exciter and as a signal receiver. In this case the single transducer element excites vibrations in the cantilever beam of the transducer device 10 using a swept frequency excitation signal delivered via the cable 20 and also responds to the excitation signal by exhibiting frequency-dependent variations in its electrical impedance. The instrument 100 measures the variation in impedance as a function of frequency by measuring the current taken for a given applied voltage. The electrical impedance will typically reach a minimum value when a resonance of the combined structure comprising the cantilever beam and the dental implant is excited. Hence the response of the dental implant to the excitation signal can be obtained by analysing the electrical impedance of the transducer element as a function of the excitation frequency. The variations in the electrical impedance of the transducer element are monitored and analysed by the instrument 100 to determine the resonant frequency.

The cable 20 is capable of supplying an excitation to the exciter transducer 50 via a connection 24 and the received signal from the transducer can be fed along the connection 22 and via the cable 20 to the instrument 100. The cable 20 and the connections 22 and 24 are arranged with respect to the cantilever beam such that any damping effect they might have on the resonant structure is reduced. The transducer device can be connected to the instrument by inserting the transducer plug 80 into an appropriate socket on the instrument 100.

The transducer plug 80 contains the EPROM device 90. FIG. 4B is a schematic diagram of the EPROM device 90 and its interface with the instrument 100. The EPROM device comprises an EPROM memory 92, an interfacing circuit 94 and a serial connector 96. The serial connector couples the EPROM device 90 to the microprocessor 110 of the instrument via a bus 98 such that the instrument can read from and write to the EPROM memory.

The EPROM device can be used to store the calibration parameters appropriate for the calculation of an ISQ value. Even slight geometrical differences due to variability in the manufacturing process of the transducer devices can result in differences in resonant frequency measurements made using different transducer devices on implants of substantially equal bone/implant interface stiffness. For this reason it is advantageous to calibrate each transducer device independently and store the appropriate calibration parameters on the EPROM 90. The calibration is typically performed during the manufacturing process. Storage of calibration parameters on the EPROM reduces the likelihood of misinterpretation of implant stability measurement results. The calibration process is performed under controlled conditions during manufacture hence the calibration parameters of a given transducer device are less likely to be incompatible with those of other transducer devices. Since the calibration values are stored on the transducer assembly itself rather than being stored separately, the calibration parameters appropriate to the wrong transducer devices are less likely to be used in the implant stability calculation.

The EPROM can also be used to store information to identify the transducer device to which it is attached such a serial number and/or a transducer type information. Furthermore, the EPROM can be used to store a limited amount of measurement data and the time and date at which the measurement was made. The time and date is obtained from a clock in the instrument. This has the advantage of reducing the likelihood of the clinician confusing measurements made on different implants in the same patient or on different patients.

The accuracy of the calibration parameters specific to a particular transducer device is important to reduce the likelihood in errors of implant stability diagnosis. The comparison of ISQ measurements on a particular bone implant is typically made several times for each patient over an extended time period. The reliability of this comparison depends upon the reliability of the calibration parameters of the transducer device used in each case. The reliability of the calibration parameters will typically diminish over time due to wear and tear of the transducer device in clinical use. Furthermore the transducer devices are typically autoclaved between patients for sterilisation. The autoclaving process can cause damage to and hence alter the frequency response of components of the transducer device. Due to the decline in accuracy of the calibration parameters as the number of uses increases, the lifetime over which the transducer is reliable for use in clinical diagnosis is limited.

Clinicians may be tempted to use a transducer device beyond the recommended maximum number of uses for economic reasons. To prevent the use of a transducer beyond the recommended lifetime, the EPROM 90 in the transducer plug 90 can be used to store and increment a counter to keep track of the number of uses of the transducer device. The counter is incremented each time the transducer plug is connected to the instrument 100. The time of each connection can also be obtained from a clock in the instrument 100 and stored on the EPROM. The counter can be incremented on the condition that the time since the last connection exceeds a predetermined minimum time since the last connection. When the counter exceeds a predetermined maximum number of uses, program code stored either on the EPROM 90 or on the instrument 100 is operable to disable the transducer device so that no further measurements can be made with it. The expired transducer device can be recalibrated by the manufacturer by erasing and reprogramming the EPROM.

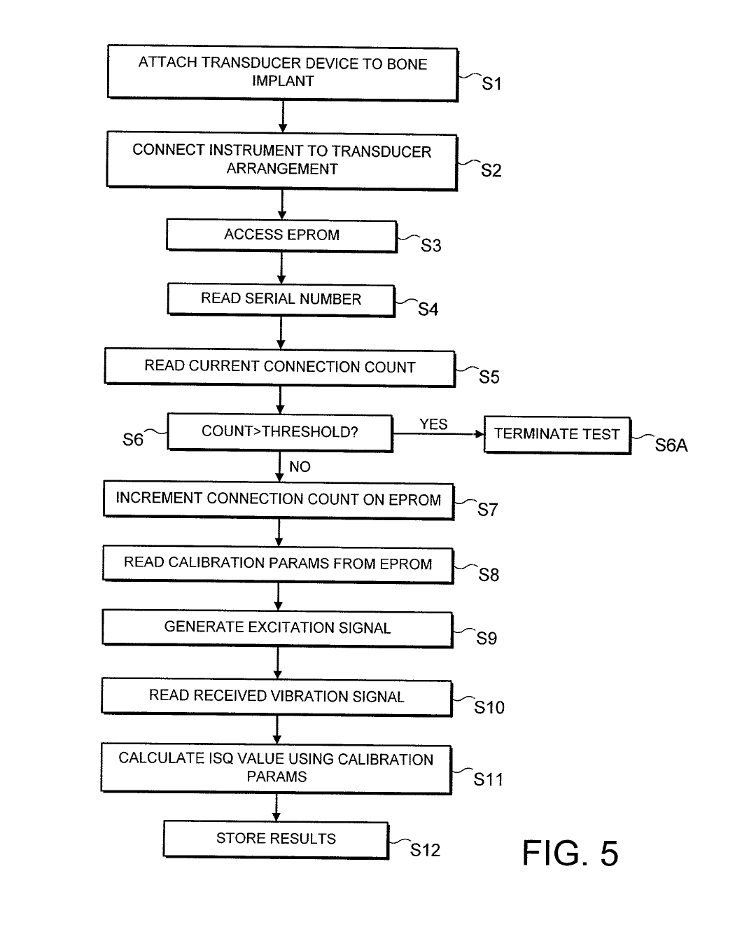

FIG. 5 is a flow diagram that demonstrates an example implant stability measurement process. At step S1 the transducer device 10 is attached to a bone implant. At step S2, the transducer device 10 is connected to the instrument 100 via the transducer plug 80.

At step S3, a memory reading circuit in the processor 110 of the instrument 100 accesses the EPROM via the interfacing circuit 94. At stage S4, the serial number which identifies the transducer device 10 is read by the instrument 100. At stage S5, the instrument reads the current connection count. The date and time at which the count was last incremented can also be read from the EPROM.

At stage S6, the connection count is compared with a predetermined threshold. If the connection count is found to be greater than the predetermined threshold then the process proceeds to stage S6A whereupon the implant stability test is terminated. The termination of the test at stage S6A occurs because the transducer device 10 has calibration parameter(s) which have expired. This reduces the likelihood of an erroneous implant stability measurement being obtained. If the connection count is less than or equal to the predetermined threshold at stage S6, the process proceeds to stage S7, where the instrument increments the connection count on the EPROM of the transducer assembly and records a connection date and time. The connection count can be incremented in dependence upon the time difference between the time associated with current connection and the last time the connection count was incremented.

At stage S8, the instrument 100 reads one or more calibration parameters from the EPROM. At stage S9, the frequency source 120 of the instrument generates a swept frequency excitation signal and delivers this signal to the exciter transducer 50. The excitation signal induces vibrations in the cantilever beam of the transducer and in the bone implant to which it is attached. The receiver transducer picks up the vibration response of the cantilever beam to the swept frequency excitation signal and generates a received vibration signal. At stage S110, the received vibration signal is read by the instrument.

At stage SI 1, the instrument analyses the received vibration signal and uses the calibration parameters read at stage S8 to calculate an ISQ value. At stage S12 the calculated ISQ value is be displayed on the LCD screen 140 of the instrument and is written to the memory 150 in the instrument and/or to the EPROM of the transducer assembly.

Although illustrative embodiments of the invention have been described in detail herein with reference to the accompanying drawings, it is to be understood that the invention is not limited to those precise embodiments, and that various changes and modifications can be effected therein by one skilled in the art without departing from the scope and spirit of the invention as defined by the appended claims.

We claim:

1. A transducer assembly for use with an apparatus for detecting the stability of a bone implant, said transducer assembly comprising:
one or more transducers operable to excite vibrations in said bone implant and to detect a response to said vibrations;
a coupling for detachably connecting said one or more transducers to circuitry for driving said vibrations and detecting said response; and
a memory device within said transducer assembly operable to store one or more parameters to be read outside of said transducer assembly via said coupling for controlling use of said transducer assembly.

2. A transducer assembly according to claim 1, wherein said memory device is operable to store one or more calibration parameters.

3. A transducer assembly according to claim 1, wherein said memory device is operable to store information to identify said transducer assembly.

4. A transducer assembly according to claim 1, wherein said memory device is operable store a connection count specifying the number of times that said transducer assembly has been connected to said circuitry.

5. An apparatus according to claim 4, wherein said memory device stores a time value indicative of a time at which said transducer was last connected to said circuitry.

6. A transducer according to claim 5, wherein if said transducer assembly is connected to said circuitry greater than a predetermined time since a last connection then said connection count is incremented.

7. A transducer assembly according to claim 6, wherein said transducer assembly is disabled when said connection count exceeds a predetermined threshold.

8. A transducer assembly according to claim 1, wherein said one or more transducers comprises a vibration exciter that also serves as a signal receiver and operates to excite vibrations in said bone implant, to detect a response to said vibrations and to generate a vibration response signal.

9. A transducer assembly according to claim 8, wherein said vibration response signal is an electrical impedance measurement.

10. A transducer assembly according to claim 1, wherein said one or more transducers comprises a vibration exciting transducer operable to excite vibrations in said bone implant and a vibration receiving transducer operable to generate a received vibration signal.

11. An instrument for use with a transducer assembly according to claim 1, said instrument comprising:
a memory reading circuit operable to read one or more parameters from a memory device within a transducer assembly; and
a variable frequency signal source operable to drive excitation vibrations in said bone implant via said transducer assembly;
a frequency response analyser operable to analyse a received vibration signal from said transducer assembly and to generate a frequency response of said bone implant to said excitation vibrations; wherein
at least one of said variable frequency signal source and said frequency response analyser operate in dependence upon said one or more parameters.

12. An apparatus for detecting the stability of a bone implant, said apparatus comprising:
a transducer assembly for use with an apparatus for detecting the stability of a bone implant, said transducer comprising:
one or more transducers operable to excite vibrations in said bone implant and to detect a response to said vibrations;
a coupling for detachably connecting said one or more transducers to circuitry for driving said vibrations and detecting said response; and
a memory device within said transducer assembly operable to store one or more parameters to be read outside of said transducer assembly via said coupling for controlling use of said transducer assembly; and
an instrument for use with said transducer assembly, said instrument comprising:
a memory reading circuit operable to read one or more parameters from a memory device within a transducer assembly; and
a variable frequency signal source operable to drive excitation vibrations in said bone implant via said transducer assembly;
a frequency response analyser operable to analyse a received response signal from said transducer assembly and to generate a frequency response of said bone implant to said excitation vibrations; wherein
at least one of said variable frequency signal source and said frequency response analyser operate in dependence upon said one or more parameters.

13. A method for detecting the stability of a bone implant, said method comprising the steps of:
exciting vibrations in said bone implant using one or more transducers of a transducer assembly and detecting a response to said vibrations;
driving said one or more transducers and detecting said response by detachably connecting said transducer assembly to circuitry via a coupling of said transducer assembly; and
storing one or more parameters in a memory device within said transducer assembly wherein said parameters are to be read outside of said transducer via said coupling and employing said parameters to control the use of said transducer assembly.
